(19) United States
(12) Patent Application Publication
(10) Pub. No.: US 2026/0325173 A1
(43) Pub. Date: Oct. 22, 2026

(54) SALPINGECTOMY METHOD

(71) Applicant: Mark Felixovich DORFMAN, Khimki (RU)

(72) Inventors: Mark Felixovich DORFMAN, Khimki (RU); Alexander Sergeevich GASPAROV, Moscow (RU); Evgenia Vladislavovna GUBANOVA, Moscow (RU); Ekaterina Dmitrievna DUBINSKAYA, Moscow (RU); Sofya Felixovna DORFMAN, Khimki (RU); Mariam Grishaevna SAAKYAN, Dzerzhinsky (RU); Elizaveta Vladimirovna ALYOSHKINA, Moscow (RU)

(21) Appl. No.: 19/682,676

(22) Filed: May 20, 2026

Related U.S. Application Data
(63) Continuation-in-part of application No. PCT/RU2024/000351, filed on Nov. 26, 2024.

(30) Foreign Application Priority Data
Nov. 27, 2023 (RU) 2023130963

(51) Int. Cl. A61B 17/42; A61B 8/00; A61B 8/12; A61B 17/00; A61B 17/34; A61B 34/10; A61B 90/00

(52) U.S. Cl. CPC A61B 17/42; A61B 8/12; A61B 8/488; A61B 8/5223; A61B 17/3423; A61B 34/10; A61B 90/37; A61B 2017/00106; A61B 2017/3425; A61B 2017/4233; A61B 2034/105; A61B 2090/373

(57) ABSTRACT

The invention relates to surgical gynaecology. A pre-operative step is performed which includes 3D transvaginal ultrasound imaging of a fallopian tube and an ovary using colour and power Doppler flow mapping modes, and contrast magnetic resonance imaging of the pelvic organs, and a 3D model of the uterine appendages is generated, in which characteristic features of the blood supply to the fallopian tube and the ovary are identified. In an intraoperative step, a laparoscopic visual inspection of the uterine appendages is performed. An intraoperative visual image of the uterine appendages is transferred to the 3D model of the uterine appendages. The 3D model and the intraoperative data regarding the uterine appendages are combined and navigation markers are created, taking into account the characteristic features of the blood supply to the fallopian tube and the ovary. A salpingectomy is performed using the intraoperative navigation, thereby preserving the perivascular system and the ovarian reserve. The method makes it possible to identify characteristic features of the blood supply to a fallopian tube and an ovary and to perform a salpingectomy without damaging the perivascular system of the fallopian tube and the ovary or diminishing the ovarian reserve.

### CROSS REFERENCE TO RELATED APPLICATIONS

This application is a Continuation-in-Part of PCT Patent Application No. PCT/RU2024/000351 having International filing date of Nov. 26, 2024, which claims the benefit of priority of Russian Patent Application No. 2023130963, filed Nov. 27, 2023, the contents of which are all incorporated herein by reference in their entirety.

### FIELD OF THE INVENTION

The invention relates to medicine, specifically to operative gynecology, and may be used for bilateral hydrosalpinx during the reproductive period. The method involves salpingectomy via laparoscopic access, which was performed using surgical navigation based on data obtained from a preoperative 3D model, for the purpose of preparing for assisted reproductive technology protocols. This invention contributes to the preservation of ovarian reserve.

### BACKGROUND

A method for preparing and performing surgical operations on pelvic organs is known from the prior art [RU 2736800 C1, 20.11.2020], in which a software-hardware complex is used that includes the stages of creating a preoperative personalized three-dimensional mathematical and graphical model of the organs and neoplasms within them, planning and simulation of organ-preserving surgical operations, and surgical intervention with the import of the preoperative three-dimensional model to create an intraoperative model. Moreover, during the surgical planning stage, the patient undergoes an ultrasound examination (US) or computed tomography (CT) or magnetic resonance imaging (MRI), and a preoperative three-dimensional model of the organs and neoplasms within them is generated, along with a surgical plan incorporating surgical navigation. Next, during the surgical operation, the preoperative model is adapted to the intraoperative parameters of the organs and tumors, and an intraoperative model with surgical navigation is generated. During the stage of constructing the preoperative model of organs and the neoplasms within them, modeling of the neoplasms in the organs is performed, focusing on aspects of interest to the surgeon in the planned operation. A preliminary surgical plan is then formed, designating navigation markers for making an incision and removing the neoplasm with minimal trauma to healthy organ tissue. Following this, an organ-preserving laparoscopic operation is performed, which begins with an assessment of the topography and dimensions of the organs and the tumors within them, and the adaptation the preoperative 3D model to the intraoperative parameters of the organs and tumors, after which, using a software-hardware complex, the video-endoscopic system's monitors provide the ability to assess the tumor's three-dimensional volume in real time, with visualization of invisible subtissue structures and vessels. Afterward, navigation markers appear on the adapted 3D model, which are used to position the incision site; the tumor is then removed from the organ, and during surgical intervention, in the event of spatial displacement of organs or changes in their geometry due to the removal of neoplasms, the model is re-aligned automatically at fixed time intervals, or manually using a computer control system. The invention provides the ability to remove neoplasms from pelvic organs with minimal trauma to healthy organ tissue, requiring no or minimal use of coagulation techniques to stop bleeding. In operation on ovary, a technical result is also the preservation of healthy ovarian tissue and ovarian reserve.

The claimed invention differs from the described one in that it involves mapping the vascular system of the adnexa that is not affected by oncological changes, identifying the vascular zones supplying blood to the fallopian tubes and ovaries in the mesosalpinx and mesovarium regions, and performing a salpingectomy while preserving the zones ovarian blood supply, which helps preserve the ovarian reserve.

A method for performing surgical intervention is also known from the prior art for the treatment of ectopic pregnancy [Laparoscopy in Gynecology/Ed. by G. M. Savelieva, Academician of the Russian Academy of Medical Sciences.—Moscow: GEOTAR, Medicine, 2000.—324 pp.], in which laparoscopic surgery is performed under ultrasound guidance, during which the mesosalpinx region is coagulated simultaneously in the anterior and posterior layers, parallel to the longitudinal axis of the fallopian tube using a bipolar coagulator without identification of vascular zones, followed by transection at the site of coagulation with scissors.

The present invention differs from the prior art in that the surgical operation maximally preserves the blood supply to the ovarian region. The anterior and posterior layers of the mesosalpinx in the avascular zone closest to the fallopian tube are dissected under intraoperative navigation using sharp and blunt techniques with the blades of endoscopic scissors. The perivascular complex is displaced using blunt dissection from the anterior layer away from the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag.

The technical result consists in expanding the arsenal of techniques and methods for salpingectomy while preserving ovarian reserve.

### SUMMARY OF THE INVENTION

The invention relates to surgical gynaecology. A pre-operative step is performed which includes 3D transvaginal ultrasound imaging of a fallopian tube and an ovary using colour and power Doppler flow mapping modes, and contrast magnetic resonance imaging of the pelvic organs, and a 3D model of the uterine appendages is generated, in which characteristic features of the blood supply to the fallopian tube and the ovary are identified. In an intraoperative step, a laparoscopic visual inspection of the uterine appendages is performed. An intraoperative visual image of the uterine appendages is transferred to the 3D model of the uterine appendages. The 3D model and the intraoperative data regarding the uterine appendages are combined and navigation markers are created, taking into account the characteristic features of the blood supply to the fallopian tube and the ovary. A salpingectomy is performed using the intraoperative navigation, thereby preserving the perivascular system and the ovarian reserve. The method makes it possible to identify characteristic features of the blood supply to a fallopian tube and an ovary and to perform a salpingectomy without damaging the perivascular system of the fallopian tube and the ovary or diminishing the ovarian reserve.

### DETAILED DESCRIPTION OF INVENTION

The present invention relates to a method of salpingectomy, in which a preoperative stage is performed, comprising: transvaginal 3D ultrasound in color and energy Doppler mapping modes to determine the size, location, and blood supply characteristics of the fallopian tube and ovary, and magnetic resonance imaging with contrast of the pelvic organs to determine the size, location, and blood supply characteristics of the fallopian tube and ovary; creation of a 3D model of the uterine appendages with determination of the characteristics of blood supply to the fallopian tubes and ovaries; the intraoperative stage, including the creation of a pneumoperitoneum with monitoring of the insufflated gas at a pressure of 12-13 mmHg; position port No. 1 (10 mm) 2 cm above and 2 cm to the left of the umbilicus along the left medio-clavicular line; place the patient in the Trendelenburg position; at points Mac-Burnea on the left, port No. 2 (5 mm) is placed; on the right, port No. 3 (5 mm) is placed; and an additional port No. 4, 5 mm, 3 cm above the line drawn between the McBurney points and 3 cm to the right of the line between the umbilicus and the line connecting the McBurney points; laparoscopic visual assessment of the uterine adnexa using 30 degrees optics; conversion of the intraoperative visual image of the uterine adnexa into a 3D model of the uterine adnexa; comparison of the 3D model with intraoperative data on the uterine adnexa and creation of navigation markers taking into account the characteristics of the blood supply to the fallopian tube and ovary; performing a salpingectomy with intraoperative navigation without damaging the perivascular complex while preserving the ovarian reserve, with the surgeon working through ports 2 and 4, an assistant working through ports 1 and 3 are used by the assistant; atraumatic clamps are inserted into ports 2 and 3; depending on the stage of the operation, an instrument for bipolar or argon or laser coagulation, or endoscopic scissors, is inserted into port 4; then the left fallopian tube is removed: using an atraumatic clamp from port No. 3, the tube is mobilized in the proximal segment at a distance of 1 cm from the uterus; using a similar clamp from port No. 2, the left fallopian tube is mobilized at a distance of 2 cm from the fimbrial segment, the surgeon and assistant simultaneously pull the fallopian tube to tension the mesosalpinx; then, under intraoperative navigation, the anterior and posterior layers of the mesosalpinx in the avascular zone as close as possible to the fallopian tube are dissected with a sharp and the tissue is dissected bluntly using the blades of endoscopic scissors; then, the perivascular complex is bluntly displaced along with the anterior layer from the area of the fallopian tube toward the ovary, followed by dissection of the posterior layer of the mesosalpinx as close as possible to the fallopian tube, followed by coagulation and transection of the uterine angle of the fallopian tube, after which the tube is removed from the abdominal cavity into an endobag; then the right tube is removed: using an atraumatic clamp from port No. 2, the tube is mobilized in the proximal segment at a distance of 1 cm from the uterus; using a similar clamp from port No. 3, the right fallopian tube is mobilized at a distance of 2 cm from the fimbrial segment; simultaneously, the surgeon and assistant pull the fallopian tube to tension the mesosalpinx; then, the anterior and posterior layers of the mesosalpinx in the avascular zone as close as possible to the fallopian tube are dissected under intraoperative navigation using sharp and blunt techniques with the blades endoscopic scissors, followed by blunt displacement of the perivascular complex from the area of the fallopian tube toward the ovary, then dissection of the posterior layer of the mesosalpinx as close as possible to the fallopian tube, followed by coagulation and transection of the uterine angle of the fallopian tube, after which the tube is removed from the abdominal cavity in an endobag.

Case 1. Patient A., 32 years old, was admitted to Gynecology Department No. 2 of the S.S. Yudin City Clinical Hospital (S.S. Yudin City Clinical Hospital) for elective surgery with the diagnosis: Infertility 1. Chronic salpingo-oophoritis with the formation of bilateral hydrosalpinx. Medical history: complaints of failure to conceive despite regular sexual intercourse over the past 2 years. One unsuccessful attempt at in vitro fertilization (IVF) and embryo transfer (ET). According to the reproductive specialist's conclusion, a repeat attempt at assisted reproductive technology (ART) is possible after removal of the fallopian tubes. Pelvic ultrasound revealed bilateral hydrosalpinx measuring 3 cm on the right and 3.5 cm on the left (ORADS 1). Somatic medical history is unremarkable. The patient underwent elective surgical treatment involving salpingectomy using the described method, which includes preoperative and intraoperative stages:

### Preoperative—First Stage:

1. Transvaginal 3D ultrasound using color and energy Doppler mapping to determine the size, location, and blood supply characteristics of the fallopian tube and ovary: Uterine body 45×40×44 mm. The myometrial structure is homogeneous ET (endometrial thickness) 5 mm, homogeneous, three-layered. Right ovary measuring 23×18×20 mm; follicular apparatus-3 follicles with diameters ranging from 3 to 5 mm are visualized in the cross-section. An enlarged fallopian tube with a diameter of up to 3 cm is visualized near the ovary, with anechoic contents and no blood flow on color Doppler imaging (CDI). The left ovary measures 25×17×22 mm, follicular apparatus in the cross-section: 3 follicles with a diameter of 4 to 6 mm are visualized. An enlarged fallopian tube with a diameter of up to 3.5 cm, anechoic contents, and 0 blood flow on CDM is visualized near the ovary. No free fluid is visualized in the pelvis. Conclusion: Echographic signs of hydrosalpinx on the right, hydrosalpinx on the left. (ORADS 1).

2. Conclusion of contrast-enhanced MRI of the pelvic organs: signs of bilateral hydrosalpinx.

3. Based on proprietary software and ultrasound and MRI data, a 3D model of the uterine appendages is developed, identifying specific features blood supply to the fallopian tube and ovary.

### Intraoperative Second Stage:

1. Creation of a pneumoperitoneum with control of the insufflated gas at a pressure of 12-13 mm Hg.

2. Placement of port No. 1. Port No. 1 (10 mm) for the laparoscope is placed 2 cm above and 2 cm to the left of the umbilicus along the left midclavicular line. The patient is positioned in the Trendelenburg position. Port No. 2 (5 mm) is placed at the left McBurney's point, Port No. 3 (5 mm) at the right McBurney's point, and an additional Port No. 4 is placed 3 cm above the line drawn between the McBurney's points and 3 cm to the right of the line connecting the umbilicus and the line joining the McBurney's points.

3. Insertion of the laparoscope (30-degree view)—laparoscopic visual assessment of the uterine adnexa: the uterine body is pink, not enlarged, and located along the midline. The right ovary is not enlarged and is in periovarian adhesions with the right fallopian tube. The right fallopian tube is dilated throughout its length to 3 cm, with peritubal adhesions to the right ovary; the fimbrial end is obliterated. The left ovary is not enlarged, with periovarian adhesions to the left fallopian tube. The left fallopian tube is dilated to 3.5 cm, with peritubal adhesions to the left ovary; the fimbrial portion is obliterated. The anterior and posterior uterine spaces are unremarkable.

4. Transfer of the intraoperative visual image of the uterine adnexa into the proprietary software.

5. Comparison of the 3D model with intraoperative data on the uterine adnexa and creation of navigation markers, taking into account the specific features of the blood supply to the fallopian tube and ovary.

6. Salpingectomy technique using the author's navigation technology without damaging the perivascular complex:

6.1. The surgeon works through ports 2 and 4, while the assistant works through ports 1 and 3.

6.2. Atraumatic forceps are inserted into ports 2 and 3. Depending on the stage of the operation, a coagulation instrument (bipolar, argon, or laser coagulation) or endoscopic scissors is inserted into port 4.

6.3. Removal of the left fallopian tube: adhesions are separated, salpingo-ovariolysis is performed on the left; using an atraumatic clamp from port No. 3, we mobilize the tube in the proximal segment at a distance of 1 cm from the uterus; using a similar clamp from port No. 2, we mobilize the left tube at a distance of 2 cm from the fimbrial segment. Simultaneously, the surgeon and assistant pull the fallopian tube to tension the mesosalpinx. The anterior and posterior layers of the mesosalpinx in the avascular zone as close as possible to the fallopian tube are dissected under intraoperative navigation using sharp and blunt techniques with the blades of endoscopic scissors. The perivascular complex is displaced using blunt dissection, starting from the anterior layer in the area of the fallopian tube toward the ovary. The posterior layer of the mesosalpinx is dissected as close as possible to the fallopian tube.

Coagulation and transection of the uterine angle of the fallopian tube. The tube is removed from the abdominal cavity in an endobag.

6.4. Removal of the right tube: adhesions are separated, and salpingo-ovariolysis on the right; using an atraumatic clamp from port No. 2, we mobilize the tube in the proximal segment at a distance of 1 cm from the uterus; using a similar clamp from port No. 3, we mobilize the right tube at a distance of 2 cm from the fimbrial segment. Simultaneously, the surgeon and assistant pull the fallopian tube to stretch the mesosalpinx. The anterior and posterior layers of the mesosalpinx in the avascular zone as close as possible to the fallopian tube are dissected using sharp and blunt techniques under intraoperative navigation endoscopic scissors. The perivascular complex is bluntly displaced from the area of the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag. Ovarian reserve markers (anti-Müllerian hormone [AMH], number of antral follicles [NAF], vascularization index [VI], blood flow index [BFI]) in patient A. before and 6 months after salpingectomy are presented in Table 1 (see graphical section).

Case 2. Patient B., 39 years old, was admitted to Gynecology Department No. 2 of the C.C. Yudin City Clinical Hospital with the diagnosis: Infertility 2. Chronic salpingo-oophoritis with formation of bilateral hydrosalpinxes.

Medical history: complaints of failure to conceive despite regular sexual intercourse without contraception for 5 years. Male factor infertility. The patient is scheduled for intracytoplasmic sperm injection (ICSI). Two unsuccessful attempts IVF and ICSI. According to the reproductive specialist's conclusion, a subsequent attempt at ART is possible after removal of the fallopian tubes. Examination revealed, based on pelvic ultrasound data from December 2022, bilateral hydrosalpinx. Referred by the reproductive specialist for salpingectomy. Menstruation since age 12, lasting 5 days, cycle every 28 days, regular, painless, moderate. Gynecological history is complicated: in 2012, laparoscopy and right cystectomy were performed. Histological examination revealed an endometrioid cyst. She has had two pregnancies: the first was a spontaneous term delivery at 39 weeks in 2008; the second pregnancy ended in a miscarriage in 2017 at 6 weeks. The patient has no significant somatic history. As part of a routine operation, the patient underwent surgical treatment involving salpingectomy using the described method, which includes preoperative and intraoperative stages:

### Preoperative—First Stage:

1. Transvaginal 3D ultrasound using color and energy Doppler mapping to determine the size, location, and blood supply characteristics of the fallopian tube and ovary: Uterine body 54×42×48 mm. The myometrial structure is diffusely heterogeneous. ET 6 mm, homogeneous, three-layered. Right ovary measuring 23×22×27 mm; follicular apparatus—in the cross-section: two follicles with diameters ranging from 3 to 5 mm are visualized. Adjacent to the ovary, an enlarged fallopian tube with a diameter of up to 3.5 cm is visualized, containing anechoic material and showing no blood flow on Doppler. The left ovary measures 34×21×30 mm; the follicular apparatus shows 5 follicles with diameters ranging from 3 to 5 mm in the cross-section. An enlarged fallopian tube with a diameter of up to 2.5 cm, anechoic contents, and 0 blood flow on T2-weighted images is visualized near the ovary. No free fluid is visualized in the pelvis. Conclusion: Echographic signs of bilateral hydrosalpinx. (ORADS 1). Adenomyosis.

2. Conclusion of contrast-enhanced MRI of the pelvic organs: signs of bilateral hydrosalpinx, diffuse changes in the myometrium.

3. Based on proprietary software and ultrasound and MRI data, a 3D model of the uterine appendages is developed, identifying the characteristics of the blood supply to the fallopian tubes and ovaries.

### Intraoperative Second Stage:

1. Creation of a pneumoperitoneum with monitoring of the insufflated gas at a pressure of 12-13 mm Hg

2. Placement of Port No. 1. Port No. 1 (10 mm) for the laparoscope is placed 2 cm above and 2 cm to the left of the umbilicus along the left midclavicular line. The patient is positioned in the Trendelenburg position. Port No. 2 (5 mm) is placed at the left McBurney's point, Port No. 3 (5 mm) at the right McBurney's point, and an additional Port No. 4 is placed 3 cm above the line drawn between the McBurney's points and 3 cm to the right of the line connecting the umbilicus and the line joining the McBurney's points.

3. Insertion of the laparoscope (30-degree view)—laparoscopic visual assessment of the uterine adnexa: the uterine body is pink, not enlarged, and located along the midline. The right ovary is not enlarged and is in periovarian adhesions with the right fallopian tube. The right fallopian tube is dilated throughout its length to 3.5 cm, with peritubal adhesions to the right ovary and the posterior leaflet of the broad ligament of the uterus; the fimbrial end is obliterated. The left ovary is not enlarged, with periovarian adhesions to the left fallopian tube. The left fallopian tube is dilated to 2.5 cm, with peritubal adhesions to the left ovary; the fimbrial end is obliterated. The anterior uterine space is unremarkable. The posterior uterine space shows multiple planar adhesions.

4. Transfer of the intraoperative visual image of the uterine adnexa to the author's software.

5. Alignment of the 3D model with intraoperative data on the uterine adnexa and creation of navigation markers taking into account the specific blood supply of the fallopian tube and ovary

6. Salpingectomy technique using proprietary navigation technology without damaging the perivascular complex:

6.1 The surgeon works through ports 2 and 4, while the assistant works through ports 1 and 3.

6.2 Atraumatic forceps are inserted into ports 2 and 3. Depending on the stages of the operation, a coagulation instrument (bipolar, argon, laser coagulation) or endoscopic scissors is inserted into port 4.

6.3 Removal of the left fallopian tube: adhesions are separated, salpingo-ovariolysis is performed on the left; using an atraumatic clamp from port No. 3, we mobilize the tube in the proximal segment at a distance of 1 cm from the uterus; using a similar clamp from port No. 2, we mobilize the left tube at a distance of 2 cm from the fimbrial end. Simultaneously, the surgeon and assistant pull the fallopian tube to tension the mesosalpinx. The anterior and posterior layers of the mesosalpinx in the avascular zone as close as possible to the fallopian tube under intraoperative navigation, using sharp and blunt techniques with the blades of endoscopic scissors. Displacement of the perivascular complex with the anterior layer is performed using a blunt technique from the area of the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag.

6.4 Removal of the right fallopian tube: adhesions were separated, and salpingo-ovariolysis on the right; using an atraumatic clamp from port No. 2, we mobilize the tube in the proximal segment at a distance of 1 cm from the uterus; using a similar clamp from port No. 3, we mobilize the right tube at a distance of 2 cm from the fimbrial segment. Simultaneously, the surgeon and assistant pull the fallopian tube to tension the mesosalpinx. The anterior and posterior layers of the mesosalpinx in the avascular zone closest to the fallopian tube are dissected under intraoperative navigation using sharp and blunt techniques with the blades of endoscopic scissors. Blunt displacement of the perivascular from the area of the fallopian tube toward the ovary. An incision is made in the posterior layer of the mesosalpinx as close as possible to the fallopian tube. Coagulation and transection of the uterine angle of the fallopian tube. The tube is removed from the abdominal cavity into an endobag. Ovarian reserve parameters for patient B. (AMH, FSH, LH, FSH-I) before and 6 months after salpingectomy are presented in Table 2 (see the graphical section).

Case 3. Patient O., 40 years old, was admitted to Gynecology Department No. 2 of the S.S. Yudin City Clinical Hospital with a diagnosis of chronic salpingo-oophoritis with the formation of bilateral hydrosalpinx.

Medical history: complaints of a dull ache in the lower abdomen over the past year. In July 2023, she consulted a gynecologist at a women's clinic, where a pelvic ultrasound revealed bilateral hydrosalpinx. One unsuccessful attempt at IVF and IUI. According to the reproductive specialist's conclusion, a repeat attempt at ART is possible after removal of the fallopian tubes. Menstruation since age 13, lasting 7 days, cycle every 28-30 days, regular, painless, moderate. Gynecological history: in 2015, laparoscopy, fallopian tube reconstruction for infertility. There was one pregnancy in total:—1^{st} spontaneous, full-term delivery at 38 weeks in 2016. The patient has no significant somatic medical history. The patient underwent scheduled surgical treatment involving salpingectomy using the described method, which includes preoperative and intraoperative stages:

### Preoperative—First Stage:

1. Transvaginal 3D ultrasound in color and energy Doppler mapping modes to determine the size, location, and characteristics of blood supply to the fallopian tube and ovary: Uterine body 50×32×45 mm. The myometrial structure is diffusely heterogeneous. ET 5 mm, homogeneous, three-layered. The right ovary measures 25×18×20 mm; the follicular apparatus in cross-section shows 3 follicles with diameters ranging from 3 to 6 mm. Near the ovary, an enlarged fallopian tube with a diameter of up to 4 cm is visualized, containing anechoic material and showing no blood flow on Doppler. The left ovary measures 33×20×28 mm, follicular apparatus in the cross-section: 5 follicles with diameters ranging from 2 to 5 mm are visualized. Near the ovary, a dilated fallopian tube with a diameter of up to 3.2 cm, anechoic contents, and 0 blood flow on DOPD is visualized. No free fluid is visualized in the pelvis. Conclusion: Echographic signs of bilateral hydrosalpinx. (ORADS 1). Adenomyosis.

2. Conclusion of contrast-enhanced MRI of the pelvic organs: signs of bilateral hydrosalpinx, diffuse changes in the myometrium.

3. Based on proprietary software and ultrasound and MRI data, a 3D model of the uterine appendages is developed, identifying the characteristics of the blood supply to the fallopian tubes and ovaries.

### Intraoperative Stage 2:

1. Creation of a pneumoperitoneum with monitoring of the insufflated gas at a pressure of 12-13 mm Hg.

2. Placement of port No. 1. Port No. 1 (10 mm) for the laparoscope is placed 2 cm above and 2 cm to the left of the umbilicus along the left midclavicular line. The patient is positioned in the Trendelenburg position. Port No. 2 (5 mm) is placed at the left McBurney's point, Port No. 3 (5 mm) at the right McBurney's point, and an additional Port No. 4 is placed 3 cm above the line drawn between the McBurney's points and 3 cm to the right of the line connecting the umbilicus and the line joining the McBurney's points.

3. Introduction of the laparoscope (30-degree view)—laparoscopic visual assessment of the uterine adnexa: the uterine body is pink in color, not enlarged, and located along the midline. The right ovary is not enlarged and is adherent to the right fallopian tube. The right fallopian tube is dilated throughout its length to 4 cm, in peritubal adhesions with the right ovary and the posterior leaflet of the broad ligament of the uterus; the fimbrial end is obliterated. The left ovary is not enlarged, in periovarian adhesions with the left fallopian tube. The left fallopian tube is dilated to 3.2 cm, with peritubal adhesions to the left ovary; the fimbrial end is obliterated. The anterior uterine space is unremarkable. The posterior uterine space shows multiple planar adhesions.

4. Transfer of the intraoperative visual image of the uterine adnexa to the author's software.

5. Comparison of the 3D model with intraoperative data on the uterine adnexa and creation of navigation markers taking into account the characteristics of the blood supply to the fallopian tube and ovary.

6. Salpingectomy technique using the author's navigation technology without damaging the perivascular complex:

6.1 The surgeon operates through ports 2 and 4, while the assistant operates through ports 1 and 3.

6.2 Atraumatic clamps are inserted into ports 2 and 3. Depending on the stages of the operation, a coagulation instrument (bipolar, argon, or laser coagulation) or endoscopic scissors is inserted into port 4.

6.3 Removal of the left fallopian tube: adhesions are separated, salpingo-ovariolysis is performed on the left side; using an atraumatic clamp from port No. 3, we mobilize the tube in the proximal segment 1 cm from the uterus; using a similar clamp from port

No. 2, we mobilize the left tube at a distance of 2 cm from the fimbrial end. Simultaneously, the surgeon and assistant pull the fallopian tube to tension the mesosalpinx. The anterior and posterior layers of the mesosalpinx in the avascular zone as close as possible to the fallopian tube under intraoperative navigation, using sharp and blunt techniques with the blades of endoscopic scissors. Displacement of the perivascular complex with the anterior layer is performed using a blunt technique from the area of the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag.

6.4 Removal of the right tube: adhesions are separated, and salpingo-ovatiolysis on the right; using an atraumatic clamp from port No. 2, we mobilize the tube in the proximal segment at a distance of 1 cm from the uterus; using a similar clamp from port No. 3, we mobilize the right tube at a distance of 2 cm from the fimbrial segment. At the same time, the surgeon and the assistant pull on the fallopian tube to tension the mesosalpinx. The anterior and posterior layers of the mesosalpinx in the avascular zone closest to the fallopian tube are dissected using sharp and blunt techniques under intraoperative navigation with the blades of endoscopic scissors. The perivascular complex is displaced using blunt dissection from the area of the fallopian tube toward the ovary. The posterior layer of the mesosalpinx is incised as close as possible to the fallopian tube. Coagulation and transection of the uterine angle of the fallopian tube. The tube is removed from the abdominal cavity into an endobag. Ovarian reserve parameters for patient O. (AMH, FSH, LH, FSH-I) before and 6 months after salpingectomy are presented in Table 3 (see the graphical section).

Case 4. Patient G., 36 years old, was admitted to Gynecology Department No. 2 of the City Clinical Hospital C.C. Yudin Hospital with a diagnosis of: Chronic salpingo-oophoritis with the formation of bilateral hydrosalpinxes. Medical history: Failure to conceive despite regular sexual intercourse without contraception. In March 2023, a pelvic ultrasound revealed bilateral hydrosalpinxes. Two unsuccessful attempts at IVF and IUI. According to the reproductive specialist's conclusion, a subsequent attempt at ART is possible after removal of the fallopian tubes. Menstruation since age 10, lasting 4 days, cycle every 26 days, regular, painless, moderate. Gynecological history: in 2003, laparotomy and appendectomy. Total pregnancies: one—in 2015, spontaneous delivery. The patient's somatic history is uncomplicated. The patient underwent elective surgical treatment consisting of a salpingectomy using the described method, which includes preoperative and intraoperative stages:

### Preoperative—First Stage:

1. Transvaginal 3D ultrasound in color and energy. Doppler mapping modes to determine the size, location, and characteristics of the blood supply to the fallopian tube and ovary: Uterine body 52×43×45 mm. The myometrial structure is diffusely heterogeneous. ET 7 mm, homogeneous, three-layered. Right ovary measuring 35×25×30 mm; follicular apparatus—the cross-section shows 4 follicles with diameters ranging from 3 to 5 mm. Adjacent to the ovary, a dilated fallopian tube with a diameter of up to 3.6 cm is visualized, containing anechoic material and showing no blood flow during transvaginal ultrasound. The left ovary measures 26×20×25 mm; the follicular apparatus in cross-section shows 2 follicles with diameters ranging from 3 to 6 mm. Next to the ovary, an enlarged fallopian tube with a diameter of up to 3.4 cm, anechoic contents, and 0 blood flow on DCE is visualized. No free fluid in the pelvis is visualized. Conclusion: Echographic signs of bilateral hydrosalpinx. (ORADS 1). Adenomyosis.

2. Conclusion of contrast-enhanced MRI of the pelvic organs: signs of bilateral hydrosalpinx, diffuse changes in the myometrium.

3. Based on proprietary software and ultrasound and MRI data, a 3D model of the uterine appendages is developed, identifying the characteristics blood supply to the fallopian tubes and ovaries.

### Intraoperative Second Stage:

1. Creation of pneumoperitoneum with control of insufflated gas at a pressure of at least 12-13 mm Hg.

2. Placement of Port No. 1. Port No. 1 (10 mm) for the laparoscope is placed 2 cm above and 2 cm to the left of the umbilicus along the left midclavicular line. The patient is positioned in the Trendelenburg position. Port No. 2 (5 mm) is placed at the left McBurney's point, Port No. 3 (5 mm) at the right McBurney's point, and an additional Port No. 4 is placed 3 cm above the line drawn between the McBurney's points and 3 cm to the right of the line connecting the umbilicus and the line joining the McBurney's points.

3. Laparoscope insertion (30-degree view)—laparoscopic visual assessment of the uterine adnexa: in the right iliac region, the sheets of the greater omentum are adherent; the uterine body is pink in color, not enlarged, and located along the midline. The right ovary is not enlarged, with periovarian adhesions to the right fallopian tube. The right fallopian tube is dilated throughout its length to 3.6 cm, with peritubal adhesions to the right ovary and the posterior leaflet of the broad ligament of the uterus; the fimbrial portion is obliterated. The left ovary is not enlarged, with periovarian adhesions to the left fallopian tube. The left fallopian tube is dilated to 3.4 cm, with peritubal adhesions to the left The fimbriated end of the ovary is fused to the posterior leaf of the broad ligament of the uterus. The anterior uterine space is unremarkable. The posterior uterine space is characterized by multiple flat adhesions.

4. Transfer of the intraoperative visual image of the uterine adnexa into the author's software.

5. Comparison of the 3D model with intraoperative data on the uterine appendages and creation of navigation markers taking into account the characteristics of the blood supply to the fallopian tube and ovary

6. Salpingectomy technique using the author's navigation technology without damaging the perivascular complex:

6.1 The surgeon operates through ports 2 and 4, while the assistant operates through ports 1 and 3.

6.2 Atraumatic forceps are inserted into ports 2 and 3. Depending on the stages of the operation, a coagulation instrument (bipolar, argon, laser coagulation) or endoscopic scissors is inserted into port 4.

6.3 Removal of the left fallopian tube: adhesions are separated, salpingo-ovariolysis is performed on the left; using an atraumatic clamp from port No. 3, we mobilize the tube in the proximal segment at a distance of 1 cm from the uterus; using a similar clamp from port No. 2, we mobilize the left tube at a distance of 2 cm from the fimbrial end. Simultaneously, the surgeon and assistant pull the fallopian tube to tension the mesosalpinx. The anterior and posterior layers of the mesosalpinx in the avascular zone closest to the fallopian tube are dissected under intraoperative navigation using sharp and blunt techniques with the blades of endoscopic scissors. The perivascular complex is displaced using blunt techniques, along with the anterior layer, from the area of the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag.

6.4 Removal of the right tube: adhesions are separated, salpingo-ovariolysis on the right; using an atraumatic clamp from port No. 2, we mobilize the tube in the proximal segment at a distance of 1 cm from the uterus; using a similar clamp from port No. 3, we mobilize the right tube at a distance of 2 cm from the fimbrial segment. Simultaneously, the surgeon and assistant pull the fallopian tube to tension the mesosalpinx. The anterior and posterior layers of the mesosalpinx in the avascular zone closest to the fallopian tube are dissected using sharp and blunt techniques under intraoperative navigation with the blades of endoscopic scissors. The perivascular complex is displaced using blunt techniques from the area of the fallopian tube toward the ovary. The posterior layer of the mesosalpinx is incised as close as possible to the fallopian tube. Coagulation and transection of the uterine angle of the fallopian tube. The tube is removed from the abdominal cavity in an endobag. The ovarian reserve parameters for patient G. (AMH, FSH, FSH, LH, FSH) before and 6 months after salpingectomy are presented in Table 4 (see the graphical section).

Case 5. Comparative characteristics of ovarian reserve parameters after laparoscopic salpingectomy using the author's method and routine techniques. In the first group (50 patients: 1a preoperative, 1b postoperative), the surgical operation was performed using the proposed method, incorporating preoperative (3D modeling) and intraoperative stages (intraoperative navigation), which allows for the identification of the specific features of the blood supply to the fallopian tube and ovary and enables salpingectomy to be performed without damaging the perivascular complex of the fallopian tube. In the second group (50 patients, 2a pre-(preoperative, 2b postoperative) salpingectomy was performed using a well established technique. The control group consisted of 30 fertile patients (with 1-2 children) of reproductive age, with regular menstrual cycles, spontaneous ovulation, and who had not used hormonal medications for 12 months, who sought follow-up care. Summary data on ovarian reserve parameters 6 months after salpingectomy are presented in Table 5 (see the graphical section).

### In the Presented Results:

1. The ovarian reserve in infertile women with tubo-ovarian formations (bilateral hydrosalpinxes) does not differ significantly from that of the control group.

2. The conventional method of salpingectomy leads to a significant decrease in ovarian reserve parameters after surgery compared to preoperative parameters.

3. The proposed method of salpingectomy includes preoperative and intraoperative stages, which allows for the identification of the characteristics of the blood supply to the fallopian tube and ovary and enables salpingectomy to be performed without damaging the perivascular complex of the fallopian tube and ovary.

Innovative modeling and navigation technologies make it possible to perform a salpingectomy without reducing the ovarian reserve (p>0.05). (57)

TABLE 1

| | Ovarian reserve parameters | | | |
|---|---|---|---|---|
| Patient A | AMH, ng/mL | AFC | VI, % | FI, 0-100 |
| Before surgery | 2.13 | 6 | 2.5 | 28 |
| After salpingectomy | 2.10 | 6 | 2.7 | 29 |
| (6 months) | | | | |

TABLE 2

| | Ovarian reserve parameters | | | |
|---|---|---|---|---|
| Patient B | AMH, ng/mL | AFC | VI, % | FI, 0-100 |
| Before surgery | 2.0 | 7 | 2.5 | 27 |
| After salpingectomy | 1.9 | 6 | 2.3 | 25 |
| (6 months) | | | | |

TABLE 3

| | Ovarian reserve parameters | | | |
|---|---|---|---|---|
| Patient O | AMH, ng/mL | AFC | VI, % | FI, 0-100 |
| Before surgery | 2.6 | 8 | 2.8 | 29 |
| After salpingectomy | 2.5 | 7 | 2.4 | 27 |
| (6 months) | | | | |

TABLE 4

| | Ovarian reserve parameters | | | |
|---|---|---|---|---|
| Patient G | AMH, ng/mL | AFC | VI, % | FI, 0-100 |
| Before surgery | 2.2 | 6 | 2.5 | 27 |
| After salpingectomy | 2.3 | 7 | 2.6 | 29 |
| (6 months) | | | | |

TABLE 5

| | Ovarian reserve parameters | | | |
|---|---|---|---|---|
| Study group | AMH, ng/mL | AFC | VI, % | FI, 0-100 |
| Group 1a | 2.22 ± 0.34 | 6.3 ± 0.8 | 2.5 ± 0.22 | 28.8 ± 1.8 |
| n-50 before | | | | |
| surgery | | | | |
| Group 1b | 2.19 ± 0.34 | 6.0 ± 0.8 | 2.3 ± 0.22 | 28.1 ± 1.8 |
| n-50 after | | | | |
| surgery | | | | |
| Wilcoxon P-value | 0.733 | 0.647 | 0.062 | 0.674 |
| Group 2a | 2.27 ± 0.3 | 7.0 ± 0.3 | 2.7 ± 0.15 | 29.2 ± 1.3 |
| n-50 before | | | | |
| surgery | | | | |
| Group 2b | 1.95 ± 0.3 | 5.1 ± 0.3 | 1.7 ± 0.15 | 26.2 ± 1.3 |
| n-50 before | | | | |
| surgery | | | | |
| Wilcoxon P-value | 0.038 | 0.007 | 0.002 | 0.0014 |
| Control group | 2.3 ± 0.3 | 6.7 ± 0.5 | 2.28 ± 0.5 | 29.1 ± 3.9 |
| n-30 | | | | |

NOTE:
No statistically significant differences were found between groups 1a, 2a and 3 (P-1a-2a-3), or between 1a and 1b (P-1a-1b) (p > 0.05).
Statistically significant differences were found between groups 2a and 2b (P2a-2b) (p < 0.05).

Clause 1. A method of salpingectomy comprising a preoperative phase and an intraoperative phase.

Clause 2. The method according to clause 1, characterized in that the preoperative stage includes transvaginal 3D ultrasound in color and energy Doppler mapping modes to determine the size, location, and blood supply characteristics of the fallopian tube and ovary, and contrast-enhanced magnetic resonance imaging of the pelvic organs to determine the size, location, and characteristics of the blood supply to the fallopian tube and ovary; creation of a 3D model of the uterine appendages with determination of the characteristics of the blood supply to the fallopian tube and ovary.

Clause 3. The method according to clause 1, characterized in that the intraoperative stage includes the creation of a pneumoperitoneum with control of the insufflated gas at a pressure of 12-13 mm Hg, the placement of a 10-mm port, 10 mm, 2 cm above and 2 cm to the left of the umbilicus along the left midclavicular line, placing the patient in the Trendelenburg position, and at the McBurney points, placing port 2 (5 mm) on the left, port 3 (5 mm) on the right, and an additional port 4 (5 mm), 3 cm above the line drawn between the McBurney points, and 3 cm to the right of the line between the umbilicus and the line connecting the McBurney points; laparoscopic visual assessment of the uterine adnexa using a 30-degree scope; transfer of the intraoperative visual image of the uterine adnexa to a 3D model of the uterine adnexa; comparison of the 3D model with intraoperative data on the uterine adnexa and creation of navigation markers taking into account the characteristics of the blood supply to the fallopian tube and ovary.

Clause 4. The method according to clause 3, characterized in that the salpingectomy with intraoperative navigation is performed without damaging the perivascular complex and while preserving the ovarian reserve, with the surgeon working through ports 2 and 4, the assistant working through ports 1 and 3, atraumatic clamps being inserted into ports 2 and 3, and an instrument for bipolar, argon, or laser coagulation, or endoscopic scissors, being inserted into port 4.

Clause 5. The method according to clause 3, wherein the left fallopian tube is removed: an atraumatic clamp is used through port 3 to mobilize the tube in the proximal segment at a distance of 1 cm from the uterus, using a similar clamp from port 2, the left fallopian tube is mobilized 2 cm from the fimbrial end; simultaneously, the surgeon and assistant pull the fallopian tube to tension the mesosalpinx, then the anterior and posterior layers of the mesosalpinx in the avascular zone are dissected under intraoperative navigation using sharp and blunt techniques with the blades of endoscopic scissors; next, the perivascular complex is displaced using a blunt technique, moving the anterior layer away from the fallopian tube toward the ovary; then the posterior layer of the mesosalpinx at the fallopian tube is incised, coagulation and transection of the uterine angle of the fallopian tube are performed, after which the tube is removed from the abdominal cavity in an endobag.

Clause 6. The method according to clause 3, characterized in that the right fallopian tube is removed as follows: using an atraumatic clamp inserted through port 2, the tube is mobilized in its proximal segment at a distance of 1 cm from the uterus; using a similar clamp inserted through port 3, the right fallopian tube is mobilized at a distance of 2 cm from the fimbrial end; simultaneously, the surgeon and assistant pull the fallopian tube to tension the mesosalpinx; then, under intraoperative navigation, the anterior and posterior layers of the mesosalpinx in the avascular zone near the fallopian tube are dissected using sharp and blunt techniques with the blades of endoscopic scissors; then the perivascular complex is displaced using blunt dissection from the area of the fallopian tube toward the ovary, followed by dissection of the posterior layer of the mesosalpinx at the fallopian tube; thereafter, coagulation and transection of the uterine angle of the fallopian tube are performed, after which the tube is removed from the abdominal cavity in an endobag.

1. A method for salpingectomy, comprising: performing a preoperative stage comprising: transvaginal 3D echography in color and energy Doppler imaging modes to determine the size, localization and features of blood supply to the fallopian tube and ovary; Magnetic resonance imaging with contrast enhancement of pelvic organs; creation of 3D model of uterine appendages with determination of uterine tube and ovary blood supply features; intraoperative stage including laparoscopic visual assessment of uterine appendages; transfer of visual intraoperative picture of uterine appendages to 3D model of uterine appendages; comparison of 3D model with intraoperative data of uterine appendages and creation of navigation marks taking into account features of blood supply of fallopian tube and ovary; performing salpingectomy with intraoperative navigation without damaging the perivascular complex while maintaining ovarian reserve.

2. A method for salpingectomy, in which a preoperative stage is performed, including: transvaginal 3D ultrasound in color and energy Doppler mapping to determine the size, location, and characteristics of the blood supply to the fallopian tube and ovary, and magnetic resonance imaging with contrast of the pelvic organs to determine the size, location, and characteristics of the blood supply to the fallopian tube and ovary; creation of a 3D model of the uterine appendages with determination of the characteristics of blood supply to the fallopian tubes and ovaries; intraoperative stage, including the creation of a pneumoperitoneum with control of the insufflated gas at a pressure of 12-13 mm Hg, placement of a 10-mm port, 10 mm, 2 cm above and 2 cm to the left of the umbilicus along the left midclavicular line, placing the patient in the Trendelenburg position; at the McBurney points on the left, port a 2.5-mm port, a 3.5-mm port on the right, and an additional 4.5-mm port, 3 cm above the line drawn between the McBurney points, and 3 cm to the right of the line between the umbilicus and the line connecting the McBurney points; laparoscopic visual assessment of the uterine adnexa using a 30-degree scope; conversion of the visual intraoperative image of the uterine adnexa into a 3D model of the uterine adnexa; Alignment of a 3D model with intraoperative data on the uterine adnexa and creation of navigation markers that account for the specific blood supply of the fallopian tube and ovary; performing a salpingectomy with intraoperative navigation without damaging the perivascular complex while preserving ovarian reserve, with the surgeon working through ports 2 and 4, the assistant working through ports 1 and 3, and 2 and 3, an instrument for bipolar, or argon, or laser coagulation, or endoscopic scissors is inserted; then removal of the left fallopian tube is performed: using an atraumatic clamp from port 3, the tube is mobilized in the proximal segment 1 cm from the uterus; using a similar clamp from port 2, the left fallopian tube is mobilized 2 cm from the fimbrial segment; simultaneously, the surgeon and assistant pull the fallopian tube to create tension the mesosalpinx, followed by the anterior and posterior layers of the mesosalpinx in the avascular zone under intraoperative navigation, using sharp and blunt techniques with the blades endoscopic scissors, followed by blunt displacement of the perivascular complex with the anterior layer from the area of the fallopian tube toward the ovary, followed by dissection of the posterior layer of the mesosalpinx at the fallopian tube, coagulation and transection of the uterine angle of the fallopian tube, after which the tube is removed from the abdominal cavity into an endobag; then the right tube is removed: using an atraumatic clamp from port 2, the tube is mobilized in the proximal segment at a distance of 1 cm from the uterus; using a similar clamp from port 3, the right fallopian tube is mobilized at a distance of 2 cm from the fimbrial segment; simultaneously, the surgeon and the assistant pull the fallopian tube to tension the mesosalpinx; then the anterior and posterior layers of the mesosalpinx in the avascular zone near the fallopian tube are dissected under intraoperative navigation using sharp and blunt techniques with the blades of endoscopic scissors; then, the perivascular complex is displaced using blunt dissection from the area of the fallopian tube toward the ovary; next, the posterior layer of the mesosalpinx at the fallopian tube is incised; following this, coagulation and transection of the uterine angle of the fallopian tube are performed, after which the tube is removed from the abdominal cavity in an endobag.
